(19) United States
(12) Patent Application Publication
(10) Pub. No.: US 2003/0139697 A1
(43) Pub. Date: Jul. 24, 2003

(54) Wound closure dressing with controlled stretchability

(76) Inventor: Thomas H. Gilman, Spring Grove, IL (US)

Correspondence Address: MARSHALL, GERSTEIN & BORUN, 6300 SEARS TOWER, 233 SOUTH WACKER, CHICAGO, IL, 60606-6357, US

(21) Appl. No.: 10/056,707

(22) Filed: Jan. 24, 2002

(51) Int. Cl. A61F 13/00; A61F 15/00

(52) U.S. Cl. 602/54

(57) ABSTRACT

A stretchable and contractible wound closure dressing is disclosed having an adhesive bodyside layer preferably formed of an hydrocolloid-containing liquid-absorbing adhesive material layer, a vapor-permeable elastomeric backing layer secured to one surface of said adhesive layer, and a multiplicity of flexible but essentially non-stretchable reinforcing members extending in spaced parallel relation across said dressing for preventing stretching of said dressing only in directions parallel with said members.

### BACKGROUND AND SUMMARY OF THE INVENTION

It is a common practice to use sutures or staples to hold the margins of a wound, particularly a surgical incision, in closed condition for healing, and it is also well known to place flexible but non-stretchable adhesive strips across such a wound to perform the same function in lieu of sutures or staples or to supplement the holding action of such sutures or staples. Such strips are ordinarily applied in laterally-spaced relation across the incision or other wound to permit limited stretching of the skin along the length of the wound while restraining such action in transverse directions parallel with the strips.

Hydrocolloid-containing wound dressings are also widely known. Such dressings ordinarily consist of a layer of moisture-absorbing and swellable hydrocolloid-containing adhesive material backed by a vapor-permeable and preferably stretchable plastic film. In such a dressing the adhesive layer generally has a continuous phase composed of a tacky, elastic or viscoelastic adhesive material such as polyisobutylene and a discontinuous phase of moisture-absorbing and swellable particles of hydrocolloid materials, such as gelatin, pectin, and/or carboxymethylcellulose. Such hydrocolloid-containing adhesive materials are often referred to as skin barrier compositions and are used not only for wound dressings but for other related products such as the adhesive wafers of ostomy appliances.

An important aspect of this invention lies in a moisture vapor diffusable wound-protective adhesive dressing, preferably one in which the adhesive layer is formed of an exudate-absorbing hydrocolloid-containing material, and which also functions as a wound-closure dressing, allowing limited stretching and contraction in all directions except in selected directions extending across the incision or other wound.

The dressing includes an adhesive layer and an elastomeric backing layer, both being moisture transmissible and preferably being sufficiently clear to permit a wound to be viewed through such layers. A multiplicity of spaced, parallel reinforcing members extend across the dressing and are secured to the backing layer and/or the adhesive layer, preferably the adhesive layer, to prevent stretching of the dressing in directions parallel with the members while allowing limited expansion and contraction of the dressing in all other directions. To achieve such purposes, the members must be flexible but non-stretchable. They are preferably but not necessarily transparent or translucent.

The non-stretchable reinforcing members may be in the form of parallel and laterally-spaced groups of parallel filaments or parallel and laterally-spaced strips. Groups of filaments are preferred for a number of reasons and, in such an embodiment, the filaments of each group are ideally arranged in parallel relation along a single plane to provide a grouping that is relatively flat and coplanar with the dressing in its entirety.

Other features, advantages and objects of the invention will become apparent from the specification and drawings.

### DRAWINGS

FIG. 1 is a perspective view of a hydrocolloid-containing wound closure dressing embodying the present invention.

FIG. 2 is an enlarged fragmentary sectional view taken along line 2-2 of FIG. 1.

FIG. 3 is a sectional view similar to FIG. 2 but illustrating a second embodiment of the invention.

FIG. 4 is a sectional view of a third embodiment of the invention.

FIG. 5 is a sectional view of a fourth embodiment of the invention.

### DETAILED DESCRIPTION OF PREFERRED EMBODIMENTS

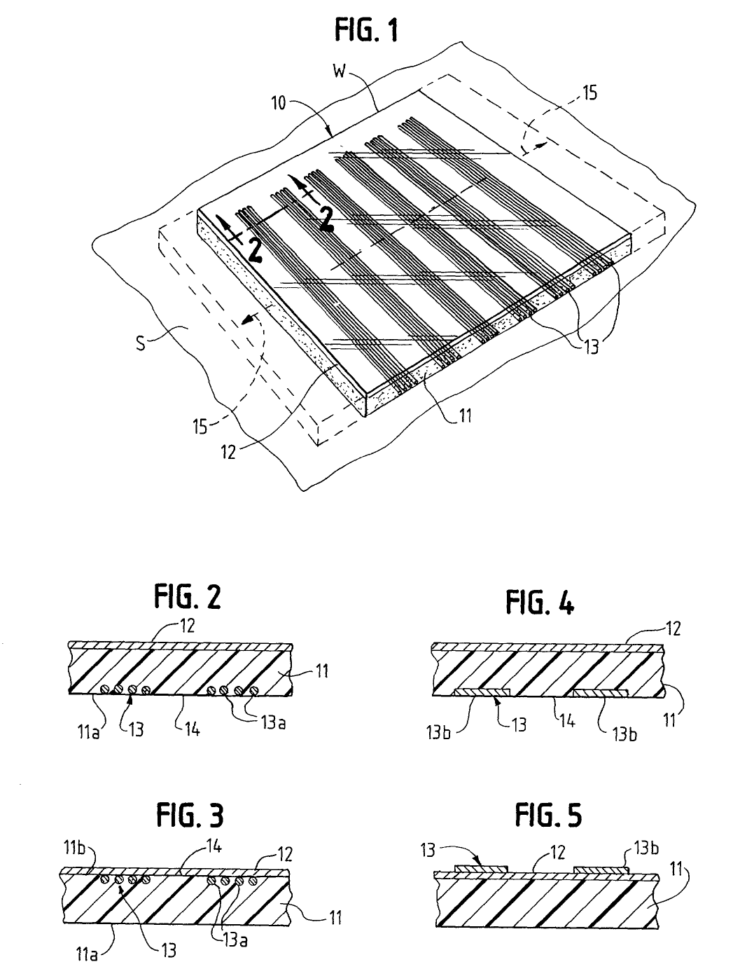

FIG. 1 illustrates a dressing 10 that embodies the present invention and that overlies a surgical wound site in which a wound, usually in the form of a surgical incision, extends along a patient's skin. Incision W may be held in closed condition by means of sutures or staples; however, an important aspect of this invention lies in the fact that the dressing itself is capable of holding the edges of the incision together because of the selectively controlled extensibility of the dressing. The discomfort produced by sutures or staples, the puncture wounds they tend to create, and the discomfort they often produce upon removal, can thereby be avoided by the dressing of this invention. While wound W is shown and described as being a surgical incision, it is to be understood that the dressing may also be used with cuts and other types of wounds that are not intentionally formed in connection with medical procedures.

The dressing 10 is shown to be generally rectangular in shape, although that configuration is not critical here. It is necessary, however, that the dressing be large enough, or that at least the combination of a plurality of such dressings disposed in contiguous or overlapping relation be large enough, to overlie not only the wound area, so that the full length of the incision or other wound is covered, but that it also extends over healthy skin areas surrounding the wound.

The dressing is generally planar and includes a bodyside layer 11 preferably composed of a hydrocolloid-containing adhesive composition commonly known in the medical field as a hydrocolloid skin barrier composition. Such a composition has both dry and wet tack, is moisture absorbent, and swells as it becomes hydrated. Such skin barrier compositions are generally soft, stretchable, pliant and conformable. They are generally known to be skinfriendly, meaning that their adhesiveness becomes less aggressive upon hydration, allowing such a dressing to be peeled or removed from the skin with only minimal discomfort and skin damage or irritation.

The preferred adhesive material of the bodyside layer 11 may be any of a variety of hydrocolloid-containing adhesive compositions well known in the art. In general, such material comprises a tacky semi-solid binder in which particles of one or more hydrocolloids are dispersed. Styrene-block polymer rubbers are preferred as the binder, but other viscous semi-solid adhesive materials may be used such as, for example, polyisobutylene, silicone rubber, acrylonitrile rubber, polyurethane rubber and natural rubber (caoutchouc). The hydrocolloid content is commonly a blend of hydrocolloids such as carboxymethylcellulose, pectin, guar gum and/or gelatin, but other hydrocolloids such as karaya, polyvinyl alcohol, carbowax and carboxypolymethylene have been disclosed in the literature and may be used. For further information concerning such hydrocolloid-containing adhesive compositions, and citations of other background materials relating thereto, reference may be had to co-owned U.S. Pat. Nos. 4,738,257, 5,492,943, and 5,935,363, the disclosures of which are incorporated herein by reference.

While the preferred adhesive material of layer 11 has been described herein as a hydrocolloid-containing adhesive, other pressure-sensitive adhesive compositions believed to be somewhat less satisfactory for this application may also be used. Layer 11 may, for example, be composed of a medical-grade acrylic adhesive which, although incapable of absorbing wound exudate and swelling as it does so, may nevertheless be applied or formed so that it is capable of transmitting moisture vapor therethrough. Such transmission may result from the permeability of the acrylic adhesive layer because of micropores formed therein or as a result of the capability of the adhesive layer to allow the diffusion of gas therethrough.

The dressing also includes a protective backing layer 12 which extends over that surface of the dressing opposite from its bodyside surface. The backing layer is preferably in the form of a thin elastomeric film having moisture vapor transmission characteristics generally approximating those of healthy skin. A polyurethane film in the thickness range of about 1 to 2 mils is believed to meet such requirements, but other polymeric films such as copolyesters or copolyamides may be used if they have similar permeability characteristics. Alternatively, the backing may be in the form of microporous fabric, such as a microporous nonwoven fabric. Vapor-permeable films are preferred because their permeability is by difusion, thereby assuring that the dressing itself is an effective barrier to liquids and pathogens, including bacteria and viruses, despite the fact that moisture accumulated by the hydrocolloid-containing layer 11 is allowed to escape by way of evaporation and difusion through the permeable backing layer.

The backing layer 12 may be secured to the hydrocolloid-containing layer 11 by means of the adhesiveness of the latter, but the security of attachment may be increased by interposing a porous or microporous medical-grade adhesive, such as a pressure-sensitive acrylic adhesive, between the two. In such a case, the adhesive should be applied or formulated in any of a variety of known ways so that it does not block the transmission of moisture through the backing layer.

Both the hydrocolloid-containing layer 11 and the backing layer 12 should be stretchable, contractible, and sufficiently transparent to permit visual inspection of the wound site through the dressing. Transparent films of polyurethane and other polymers having similar properties are known, and while skin barrier layers of a substantial thickness tend to be opaque, it has been found that if their thickness falls within the general range of 10 to 45 mils they will be sufficiently transparent to allow at least limited wound inspection. Skin barrier materials of even greater thickness may be formulated with sufficient clarity to permit visual inspection of a wound.

It is known that hydrocolloid adhesives tend to become more adhesive during the initial stages of exudate absorption but then tend to lose much of their adhesiveness as saturation is reached. In the use of the dressing of this invention, saturation over a wound is unlikely to occur because surgical incisions and other cuts tend to be relatively low in exudate discharge and because the backing layer permits fluid evaporation. Also, since the dressing is substantially larger than a wound site, with the outer portions of the dressing being in contact with healthy skin, full hydration of the skin-contacting portions of the dressing would be unlikely to occur even if exudate should cause skin barrier saturation in the immediate vicinity of the wound.

Both the hydrocolloid-containing layer 11 and the backing layer 12 are stretchable in all directions in the plane of the dressing in the absence of means specifically limiting such stretchability. In the dressing of this invention, such means takes the form of a multiplicity of laterally-spaced reinforcing members 13 of flexible but non-stretchable material. The reinforcing members may comprise a plurality of flat strips of non-stretchable polymeric film, such as a polyester film, or a fabric or film having longitudinal stretch-resisting fibers, but other materials having similar properties may be used.

In a preferred embodiment, each member consists essentially of a grouping of parallel, stretch-resistant filaments or fibers 13a in which the filaments of each grouping may or may not be connected to each other. The fibers of each group should lie alongside each other in the same plane as the dressing, with each group being spaced laterally from adjacent groups in the series. Ideally, the filaments or strips are sufficiently transparent that a wound site may be inspected through them, but even where such filaments or strips are relatively opaque, visual inspection is still possible because of the spaces provided between the strips or groups of filaments.

In the embodiments of FIGS. 1 and 2, the reinforcing members 13 constitute groups of filaments 13a, with the filaments of each group extending along the underside 11a of adhesive layer 11. The filaments may be embedded in the adhesive material so that each group has its undersurface flush with the undersurface 11a of the adhesive layer, although such embedding may not be essential if the filaments are of sufficiently small diameter. The groups of filaments are arranged in parallel with spaces 14 extending therebetween. The adhesive material layer 11 is fully exposed along spaces 14 for securely adhering the dressing to a patient's skin S on opposite sides of an incision or other wound, with the filaments extending generally across or at right angles to the wound.

If desired, a suitable pressure-sensitive adhesive may be applied to and extend along the underside of the groups of filaments or, alternatively, the groups of filaments may be more completely imbedded in adhesive layer 11 so that the undersurface 11a of the adhesive layer is uninterrupted by the filament groups. It has been found, however, that the arrangement depicted in FIGS. 1 and 2 has the advantage of simplicity and that the adhesive contacting the skin in the spaces between the groups of filaments provides sufficient adhesion for maintaining the dressing in place while resisting forces tending to draw the margins of a wound apart.

FIG. 3 depicts a second embodiment in which the reinforcing members 13 are also in the form of spaced parallel groups of filaments 13a but such groups extend over the generally planar upper surface 11b and are located between adhesive layer 11 and backing layer 12. Such an arrangement has the advantage of providing an uninterrupted adhesive surface 11a in contact with the wound site, but that advantage may be offset to a limited extent by the fact that the filaments extend along a plane that is spaced away from the surface of the skin.

In a third embodiment illustrated in FIG. 4, the arrangement is essentially the same as the embodiment of FIGS. 1 and 2 except that the reinforcing members 13 take the form of flat strips 13b of flexible but nonstretchable material as previously described. Again, while the undersurfaces of the strips might be coated with a pressure-sensitive adhesive, or might be completely imbedded within adhesive layer 11, such variations are not necessary because adequate adhesion between the adhesive layer and the skin is achieved along the spaces 14 between the reinforcing strips.

In the fourth embodiment depicted in FIG. 5, the reinforcing members 13 are again in the form of spaced parallel strips 13b, but such strips are located above backing layer 12. The strips are secured to the surface of the backing layer by an adhesive or by any other suitable means.

While certain embodiments are disclosed as having reinforcing members in the form of parallel groups of filaments and others in the form of parallel strips, it is to be understood that the arrangements may be interchanged with strips being used instead of filaments, and vice versa. Also, regardless of the location of the spaced parallel reinforcing means, whether above, below or through the adhesive layer, such reinforcing members effectively prevent stretching and contracting of the dressing in directions parallel with such members while allowing limiting stretching and contracting of the dressing in all other directions. The lateral spacing between the parallel reinforcing members allows stretching and contracting of the dressing in directions transverse to those members and also permits inspection of a wound area through the dressing even if the reinforcing members are opaque. However, as already stated, it is preferred that the reinforcing members be formed of a transparent or at least translucent material.

In use, the dressing is oriented so that the nonstretchable members extend across, at generally right angles to, the elongate incision or wound W. Exudate from the wound is absorbed by the hydrocolloid-containing surface-contacting adhesive layer with such absorption causing the swelling of the barrier material in the immediate vicinity of the wound. If such localized moisture-induced swelling occurs, increased tension caused by outward bowing of the portions of the strips bridging the wound tend to pull the edges of the wound into even tighter mutual engagement.

The result is a dressing that is a barrier to liquids and pathogens and that also functions as a wound closure device. The protection afforded by such a dressing need not be interrupted for visual inspection of a wound because of the transparency of the dressing. While the non-stretchable filaments or strips prevent the edges of the wound from being pulled away from each other, that is, from being drawn outwardly in the directions of the strips that bridge the wound, the dressing nevertheless allows limited stretching action in other directions, as indicated by broken lines and arrows 15 in FIG. 1, thereby enhancing patient comfort and reducing the possibility that patient movement might exert sufficient force to disrupt the adhesive attachment between the dressing and a patient's skin.

In the embodiments illustrated, the dressings are generally planar and of uniform thickness throughout, but it is to be understood that the edges of such dressings may be tapered or beveled as disclosed, for example, in co-owned U.S. Pat. No. 5,591,447. In addition to the advantages disclosed in such patent (the disclosure of which is incorporated by reference herein), such tapering or beveling permits the edge portions of two or more of the dressings of this invention to be overlapped without producing excessive thickness in the area of overlap.

While in the foregoing, I have disclosed embodiments of the invention in considerably detail for purposes of illustration, it will be understood by those skilled in the art that many of these details may be varied without departing from the spirit and scope of the invention.

1. A wound closure dressing of controlled stretchability that is both liquid and pathogen impermeable; said dressing comprising an adhesive layer of flexible and stretchable pressure-sensitive adhesive material; a stretchable and contractible backing layer secured to one side of said adhesive layer; and a multiplicity of flexible but substantially nonstretchable reinforcing members secured to and extending in spaced parallel relation across said dressing for preventing stretching of said dressing in directions parallel with said members while allowing stretching and contraction of said dressing in all other directions.

2. The wound closure dressing of claim 1 in which said reinforcing members comprise spaced parallel flexible but substantially non-stretchable filaments.

3. The wound closure dressing of claim 2 in which said filaments are arranged in spaced parallel groups.

4. The wound closure dressing of claim 3 in which said filaments extend in parallel relation along substantially the same plane.

5. The wound closure dressing of claim 3 in which said filaments extend along a side of said adhesive layer opposite from said one side.

6. The wound closure dressing of claim 3 in which said filaments extend between said backing layer and said adhesive layer.

7. The wound closure dressing of claim 1 in which said reinforcing members comprise spaced parallel strips of flexible but substantially non-stretchable material.

8. The wound closure dressing of claim 7 in which said strips extend in parallel relation along substantially the same plane.

9. The wound closure dressing of claim 7 in which said strips extend along a side of said adhesive layer opposite from said one side.

10. The wound closure dressing of claim 7 in which said strips extend between said backing layer and said adhesive layer.

11. The wound closure dressing of claim 7 in which said strips extend across said backing layer along a surface thereof opposite from said adhesive layer.

12. The wound closure dressing of claim 1 in which said backing layer and said adhesive layer have a transparency allowing visual inspection through said dressing of a wound site covered by said dressing.

13. The wound closure dressing of claim 1 in which said adhesive layer comprises a continuous phase of an elastomeric adhesive material and a discontinuous phase of hydrocolloid particles dispersed throughout the continuous phase.
